(19) United States
(12) Patent Application Publication
(10) Pub. No.: US 2025/0213306 A1
(43) Pub. Date: Jul. 3, 2025

(54) DEVICE AND METHOD FOR ASSISTANCE IN A PERCUTANEOUS TUMOUR ABLATION PROCEDURE

(71) Applicants: Institut national de recherche en informatique et en automatique, Le Chesnay (FR); Assistance Publique - Hôpitaux De Paris, Paris (FR); Université de Bordeaux, Bordeaux (FR); Institut Polytechnique de Bordeaux, Talence (FR); Centre Nationale de la Recherche Scientifique, Paris Cedex 16 (FR)

(72) Inventors: Clair POIGNARD, Rennes (FR); Baudouin DENIS DE SENNEVILLE, Talence (FR); Olivier SEROR, Paris (FR)

(21) Appl. No.: 18/850,367

(22) PCT Filed: Mar. 22, 2023
(86) PCT No.: PCT/EP2023/057381

§ 371 Date: Sep. 24, 2024

(30) Foreign Application Priority Data
Mar. 25, 2022 (FR) 2202697

(51) Int. Cl. A61B 34/10; A61B 18/00; A61B 18/14; A61B 90/00

(52) U.S. Cl. CPC A61B 34/10; A61B 18/1477; A61B 90/37; A61B 2018/00577; A61B 2034/104; A61B 2090/3764

(57) ABSTRACT

A medical assistance device. The device includes: a memory arranged to receive pre-procedural image data of one or more area(s) of interest; a tool arranged to receive current image data of one or more area(s) of interest including at least one electromagnetic stimulation needle obtained by cone beam computed tomography during a percutaneous ablation procedure based on electromagnetic fields, to implement registration of current image data and pre-procedural image data, and to return cone beam computed tomography augmented image data; and a simulator arranged to receive cone beam computed tomography augmented image data and electromagnetic stimulation parameters and to return electromagnetic field simulation data.

### CROSS-REFERENCE TO RELATED APPLICATIONS

This Application is a Section 371 National Stage Application of International Application No. PCT/EP2023/057381 filed Mar. 22, 2023, published as WO2023/180404A1 on Sep. 28, 2023, not in English, which claims priority to French Patent Application No. FR2202697, filed Mar. 25, 2022, the contents of which are incorporated by reference herein in their entireties.

### FIELD OF THE DISCLOSURE

The invention relates to the field of interventional oncology, in particular assistance in a percutaneous tumour ablation procedure based on electromagnetic fields.

### BACKGROUND OF THE DISCLOSURE

When a tumour is detected in a patient, the latter can generally be removed by surgical ablation, so-called resection. Nonetheless, some deep tumours are not resectable such that percutaneous tumour ablation—in particular that one based on electromagnetic fields—is then an alternative of choice in a curative perspective.

Some of the known techniques involve inserting one or more needle(s) into the tumour via a percutaneous route. In the case of a radiofrequency ablation, for example, the needle is connected to a generator to conduct the electric current to the tumour and destroy it by thermal energy. It is also possible to use microwaves. Besides thermal treatments, irreversible electroporation consists in using the needles to subject the tumour to pulsed electromagnetic fields so as to damage the membrane of the tumour cells and destroy these by coagulative necrosis or apoptosis. Moreover, the increase in the permeability of the tumour cells could be exploited to deliver therapeutic products therein in the context of electrochemotherapy which consists in injecting cytotoxic molecules, after application of electrical pulses.

According to current clinical recommendations, images of the target region and of the surrounding vital organs are collected several weeks before the percutaneous tumour ablation using medical imaging techniques such as magnetic resonance imaging (MRI) or computed tomography scanning (better known by the English term “CT-scan”). The day of the ablation, the interventional radiologist inserts the needles into the tumour via a percutaneous route, and verifies positioning thereof using images obtained by one of these techniques or by cone beam computed tomography (better known by the English acronym CBCT standing for “Cone Beam Computed Tomography”). Afterwards, the electromagnetic energy is delivered to the tumour via the needles.

Nonetheless, the interventional radiologist has no visual feedback during the percutaneous ablation procedure. The effectiveness of the treatment is assessed by acquisition of monitoring images (CT-Scan and/or MRI) only a few days after the intervention for electroporation or a few weeks for radiofrequency ablation.

Each medical imaging technique (MRI, CT-scan or CBCT) has its advantages and drawbacks. Magnetic resonance imaging (MRI) and computed tomography scanning (CT-scan) provide high-quality images. Nonetheless, an MRI apparatus has a high cost, a low availability and, above all, is incompatible with metal needles whereas computed tomography scanning involves high risks of irradiation since it consists in measuring X-ray absorption by the tissues. Conversely, cone beam computed tomography is a simple, less irradiating, low-cost medical imaging technique which can be used during the intervention. Although increasingly used in operating rooms, this technique provides only low-quality images and therefore does not allow properly visualising the tumour.

### SUMMARY

The present invention improves the situation.

In this respect, the invention relates to a medical assistance device comprising:
a memory arranged so as to receive pre-procedural image data of one or more area(s) of interest,
a tool arranged so as to receive current image data of one or more area(s) of interest including at least one electromagnetic stimulation needle obtained by cone beam computed tomography during a percutaneous ablation procedure based on electromagnetic fields, to implement registration of current image data and pre-procedural image data, and to return cone beam computed tomography augmented image data, and
a simulator arranged so as to receive cone beam computed tomography augmented image data and electromagnetic stimulation parameters, and to return electromagnetic field simulation data.

In one or more embodiments, the device further comprise a screen arranged so as to receive electromagnetic field simulation data, the simulation data comprising distribution data of the electromagnetic field, and to display a distribution of the electromagnetic field in one or more area(s) of interest.

The invention also relates to a medical assistance method implemented by the previously-described device and comprising:
receiving pre-procedural image data of one or more area(s) of interest including all or part of a tumour,
receiving current image data of one or more area(s) of interest including at least one electromagnetic stimulation needle obtained by cone beam computed tomography during a percutaneous ablation procedure based on electromagnetic fields,
implementing a registration of current image data and pre-procedural image data,
returning cone beam computed tomography augmented image data,
receiving cone beam computed tomography augmented image data and electromagnetic stimulation parameters, and
returning electromagnetic field simulation data.

For example, the pre-procedural image data are obtained by magnetic resonance imaging or computed tomography scanning.

In one or more embodiments, the pre-procedural image data and the current image data relate to three-dimensional images of one or more area(s) of interest.

In one or more embodiments, the registration comprises an initial operation of implementing a nearest neighbour algorithm applied to at least one pair formed of a pre-procedural image and a current image.

Advantageously, the pre-procedural image and the current image are respectively decomposed into a set of local elements—or patches —, and the nearest neighbour algorithm comprises a propagation during which a distance between a patch of the pre-procedural image and a patch of the current image is calculated as follows:

$$D ( VP ; VC ) = - \frac{{\int }_{Γ} \vert {\vec{∇}}_{I} ( VP + r ) \cdot {\vec{∇}}_{J} ( VC + r ) \vert dr}{{\int }_{Γ} {‖ {\vec{∇}}_{I} ( VP + r ) ‖}_{2} {‖ {\vec{∇}}_{J} ( VC + r ) ‖}_{2} dr}$$

where: —I is a pre-procedural image and J is a current image,
   VP and VC are the respective centres of the patches of the pre-procedural image I and of the current image J,
   r is a local variable corresponding to a displacement vector whose variation enables VP+r and VC+r to cover the entirety of the considered patch r, and
   \vec{∇}_{I} and \vec{∇}_{J} are the respective gradients of the pre-procedural image and the current image.

In one or more embodiments, the registration comprises determining a geometric transformation allowing switching from a pre-procedural image into a current image, or vice versa, by minimising an energy function depending on an estimated criterion based on respective gradients of the pre-procedural image and of the current image. The energy function may be defined as follows:

$$E ( T ) = {\int }_{Ω} D ( T ) + \frac{α}{2} ( {‖ \vec{∇} u ‖}_{2}^{2} + {‖ \vec{∇} v ‖}_{2}^{2} + {‖ \vec{∇} w ‖}_{2}^{2} ) d \vec{r}$$

where: —T is a geometric transformation,
   E is the energy function defined on an image domain 0,
   D is the estimated criterion,
   α is a weighting coefficient,
   u, v and w are the components of the geometric transformation T.

In one or more embodiments, the energy function is minimised by solving a system of Euler-Lagrange equations obtained by deriving the energy function with respect to each component of the geometric transformation, the value of each component being calculated by iteration.

For example, the criterion is estimated as follows:

$$D ( T ) = {e}^{- C ( T )}$$

$$C ( T ) = \frac{{\int }_{Γ} \vert {\vec{∇}}_{I} ( T ( V ) ) \cdot {\vec{∇}}_{J} ( V ) \vert dV}{{\int }_{Γ} {‖ {\vec{∇}}_{I} ( T ( V ) ) ‖}_{2} {‖ {\vec{∇}}_{J} ( V ) ‖}_{2} dV}$$

where: —T is a geometric transformation,
   D is the calculated criterion on a patch ┌,
   I is a pre-procedural image and J is a current image,
   V is a sub-element of the patch ┌, and
   \vec{∇}_{I} and \vec{∇}_{J} are the respective gradients of the pre-procedural image and of the current image.

In one or more embodiments, a Sobel filter is applied to the pre-procedural image and to the current image to determine the amplitude and the orientation of their respective gradients.

In one or more embodiments, the electromagnetic stimulation parameters include the intensity of the electric current flowing in at least one electromagnetic stimulation needle, and the simulation data comprise distribution data of the electromagnetic field. The distribution of the electromagnetic field is determined from an effective electrical conductivity of tissues in one or more area(s) of interest calculated according to the intensity of the electric current flowing in the at least one electromagnetic stimulation needle.

In one or more embodiments, one or more pair(s) of electromagnetic stimulation needles are used to implement the percutaneous ablation procedure based on electromagnetic fields, so that two electromagnetic stimulation needles of the same pair act as active electrodes, and the effective electrical conductivity of the tissues is calculated as follows for such a pair of active electrodes:

$${σ}_{eff} = \frac{{I}_{measured}}{{g}^{+} {\int }_{E {L}^{+}} {\partial }_{n} {u}^{+} ( x ) d x + {g}^{-} {\int }_{E L -} {\partial }_{n} {u}^{-} ( x ) d x}$$

where: —σₑₜₜ is the effective electrical conductivity of the tissues,
   I_{measured} is the measured intensity of the electric current,
   EL⁺ and EL⁻ are the active electrodes,
   g⁺ and g⁻ are the respective electrical voltages of the active electrodes EL⁺ and EL⁻,
   x is a local variable flowing through each of the active electrodes EL⁺ and EL⁻, and
   σₙu⁺, respectively σₙu⁻, is the normal component of the gradient of the potential along the electrode EL⁺, respectively EL⁻.

The invention also relates to a computer program comprising instructions for implementing the previously-described method, when the instructions are executed by at least one processor.

### BRIEF DESCRIPTION OF THE DRAWINGS

Other features, details and advantages will become apparent upon reading the detailed description hereinafter, and from the analysis of the appended drawings, wherein:

FIG. 1 schematically illustrates a device for assistance in a percutaneous tumour ablation procedure according to the invention;

FIG. 2 illustrates a method for assistance in a percutaneous tumour ablation procedure according to the invention; and

FIG. 3 illustrates an operation of registering current image data and pre-procedural image data of one or more area(s) of interest.

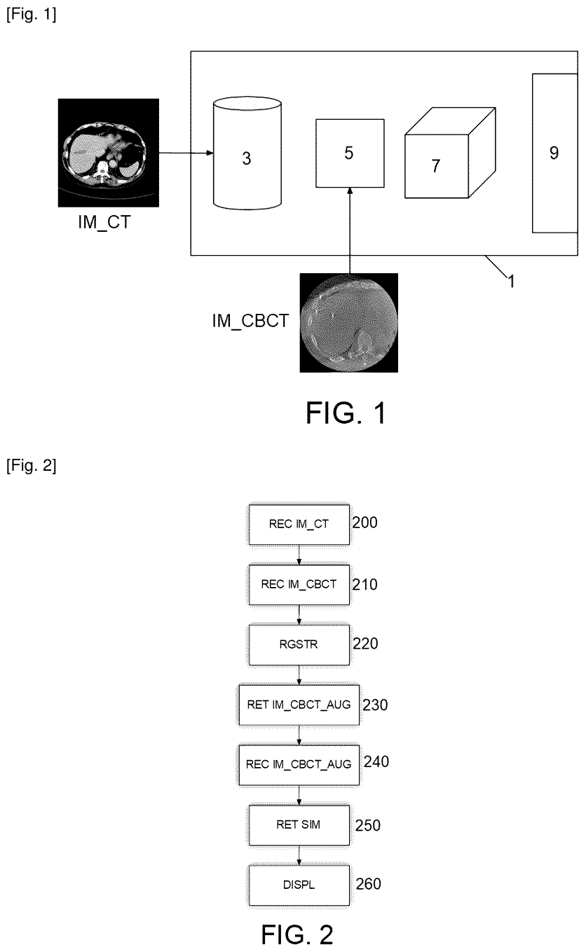

### DETAILED DESCRIPTION OF ILLUSTRATIVE EMBODIMENTS

[FIG. 1] schematically illustrates a device 1 for assistance in a percutaneous tumour ablation procedure based on electromagnetic fields, hereinafter so-called the device 1.

A percutaneous tumour ablation procedure based on electromagnetic fields is particularly suitable when the tumour of a patient is not resectable, i.e. when a surgery is not possible to remove the tumour in its entirety. Such a case may be encountered when the tumour is too deep or the patient has contraindications due to his/her state of health.

The known percutaneous tumour ablation techniques—for example radiofrequency or microwave ablation, or irreversible electroporation—consist in inserting one or more electromagnetic stimulation needle(s) into the tumour.

The device 1 comprises a memory 3, a tool 5 and a simulator 7. In the example illustrated in [FIG. 1], the device 1 also comprises a screen 9.

The memory 3 is arranged so as to receive pre-procedural image data of one or more area(s) of interest.

By “pre-procedural image”, it should herein be understood an image obtained before the percutaneous tumour ablation procedure. Hence, the pre-procedural images are obtained before the interventional radiologist proceeds with the percutaneous ablation of the tumour and, more specifically, before the insertion of any needle into the tumour of the patient.

In general, an area of interest refers to an anatomical region of the patient the visualisation of which is necessary or useful for the preparation of the operation or for the implementation thereof. For example, an area of interest includes all or part of the detected tumour to allow determining its morphological characteristics, for example its size or its position, in particular with regards to the surrounding vital organs. Nonetheless, an area of interest may also include only portions of the body affected or likely to be affected by the tumour or organs to which the interventional radiologist must lend particular attention during the percutaneous tumour ablation procedure.

The pre-procedural images of the area(s) of interest are collected up to several weeks before the surgery using medical imaging techniques. The techniques used on this occasion are magnetic resonance imaging (MRI) or computed tomography scanning (CT-scan) which have the advantage of providing high-quality images and therefore an accurate visualisation of the tumour as well as of the surrounding vital organs.

A pre-procedural image may be two-dimensional or three-dimensional.

In the example illustrated in [FIG. 1], the shown pre-procedural image IM_CT is acquired by computed tomography scanning.

The tool 5 is arranged so as to receive current image data of one or more area(s) of interest including at least one electromagnetic stimulation needle. It should herein be understood that an area of interest may comprise only one portion of an electromagnetic stimulation needle.

By “current image”, it should herein be understood an image obtained during the percutaneous tumour ablation procedure, i.e. while the interventional radiologist proceeds with the ablation of the tumour of the patient in the operating room. More specifically, a current image is an image obtained after at least one electromagnetic stimulation needle has been inserted.

Indeed, as explained before, the percutaneous tumour ablation procedure involves inserting one or more needle(s) into the tumour. Hence, compared to a pre-procedural image, a current image has the interest of visualising the position of an electromagnetic stimulation needle.

Moreover, in the context of the invention, the current images are obtained by cone beam computed tomography (CBCT). Indeed, cone beam computed tomography can be used during the intervention, unlike magnetic resonance imaging, and is less irradiating than computed tomography scanning. As explained before, medical imaging techniques such as magnetic resonance imaging and computed tomography scanning are rather suitable for acquiring pre-procedural image data.

Cone beam computed tomography consists of an X-ray emitter whose conical shaped irradiation beam allows scanning the entire volume, in one single sequence of rotation—complete (360°) or not (180°)—around the patient, and reconstructing a three-dimensional representation thereof. Conversely, computed tomography scanning is based on a flattened X-ray which only allows exploring a cut of the analysed volume.

The conical beam passes through the patient and is analysed, after attenuation, by a detector. The detector is secured to the emitter so that, during rotation, the emitter releases a pulse of X-rays which pass through the body of the patient so as to be received by the detector which simultaneously performs a rotation. In general, the sensor is a planar sensor which allows obtaining, at each angular displacement, a two-dimensional image of the volume crossed by the conical beam. Afterwards, the computed plane images—in the range of several hundreds—are processed by a volume reconstruction algorithm for a virtual visualisation of the analysed anatomical structures. A brightness amplifier may be used as an alternative to the planar sensor and has the advantage of having a very high luminous sensitivity.

In the same manner as the pre-procedural image, the current image obtained by cone beam computed tomography may be a two-dimensional image or a three-dimensional image.

A current image IM_CBCT, obtained by cone beam computed tomography, is illustrated in [FIG. 1].

Moreover, the tool 5 is arranged so as to implement a registration of current image data and of pre-procedural image data. Hence, one could understand that the tool 5 is arranged so as to communicate with the memory 3 and receive pre-procedural image data from the latter.

The image registration (often referred to by the English name “image registration”) consists in comparing images in order to match them, in particular in order to combine them or superpose them. In general, the registration involves estimating a geometric transformation enabling an optimum and coherent superposition of the common or similar features present on the processed images.

The tool 5 is herein arranged so as to receive, on the one hand, a pre-procedural image and, on the other hand, a current image to perform a registration of these images. This registration is complex because the pre-procedural images are obtained by magnetic resonance imaging or computed tomography scanning, while the current images are obtained by cone beam computed tomography.

Preferably, the pre-procedural images and the current images have the same dimension to facilitate registration.

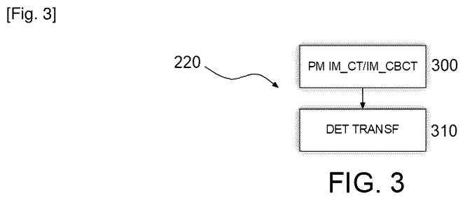

The registration implemented by the tool 5 will be detailed later on in the description, with reference to [FIG. 2] and especially to [FIG. 3].

Finally, the tool 5 is arranged so as to return cone beam computed tomography augmented image data.

The tool 5 allows generating images whose quality is close to that of the pre-procedural image while including the electromagnetic stimulation needles visible on the current images. Such images form augmented images, the visualisation of which, during the percutaneous tumour ablation procedure, allows verifying and adjusting, accurately, the position of the electromagnetic stimulation needles. The cone beam computed tomography augmented image data may be stored in the memory 3.

The tool 5 may be made in the form of an appropriate computer program executed on one or more processor(s). Such a processor may be made in any known manner, for example in the form of a microprocessor for a personal computer, a programmable logic circuit (better known by the English acronym PLD standing for “Programmable Logical Device”) or a dedicated chip of the FPGA (English acronym standing for “Field Programmable Gate Array”) or SoC (English acronym standing for “System on Chip”) type, of a computer resource grid, of a microcontroller, or of any other specific form having the computing power necessary for the implementation of the medical assistance method described hereinbelow. One or more of these elements may also be made in the form of application-specific electronic circuits such as an ASIC (English acronym standing for “Application-Specific Integrated Circuit”). A combination of processors and electronic circuits may also be considered. The tool 5 may also comprise a memory or any known type of storage media for storing instructions of a computer program, the implementation of which by the processor(s) results in the operation of the tool 5.

The simulator 7 is arranged so as to receive cone beam computed tomography augmented image data.

For example, the simulator 7 is arranged so as to communicate with the tool 5 to receive the cone beam computed tomography augmented image data, or with the memory 3 when the cone beam computed tomography augmented image data are stored in the latter.

Furthermore, the simulator 7 is arranged so as to receive electromagnetic stimulation parameters. For example, these parameters include the intensity of the electric current flowing in at least one electromagnetic stimulation needle.

From the cone beam computed tomography augmented image data and from the electromagnetic stimulation parameters, the simulator 7 is arranged so as to return electromagnetic field simulation data.

The simulator 7 allows modelling and translating in the form of data the electromagnetic field to which the area of the tumour is exposed, whether in the context of a radiofrequency or microwave ablation, an irreversible electroporation or any other percutaneous tumour ablation technique based on electromagnetic fields. Such data may be used during the surgery, for example by the interventional radiologist, to determine the distribution of the electromagnetic field generated by the electromagnetic stimulation needle(s).

Herein again, the electromagnetic field simulation data may be stored in the memory 3.

In the same manner as the tool 5, the simulator 7 may be made in the form of an appropriate computer program executed on one or more processor(s). As regards making of such a processor, the previous considerations relating to the tool 5 also apply to the simulator 7. Moreover, herein again, the simulator 7 may also comprise a memory or any known type of storage media for storing instructions of a computer program, the implementation of which by the processor(s) results in the operation of the simulator 7.

In [FIG. 1], the memory 3, the tool 5 and the simulator 7 are shown as separate elements. Nonetheless, it should herein be understood that this distinction is functional and that, consequently, all or part of these three elements could consist of the same element adapted to implement the associated functionalities.

Finally, the screen 9 is arranged so as to receive electromagnetic field simulation data. For example, the screen 9 is arranged so as to communicate with the simulator 7 to receive the electromagnetic field simulation data, or with the memory 3 when the electromagnetic field simulation data are stored in the latter.

The screen 9 is arranged so as to display a distribution of the electromagnetic field in one or more area(s) of interest. The screen 9 enables the interventional radiologist to directly visualize not only the position of the electromagnetic stimulation needles but also the distribution of the electromagnetic field to which the tumour is exposed.

The screen 9 is optional and may be directly integrated into the device 1 or be remtoe from the latter.

A method for assistance in a percutaneous tumour ablation procedure will now be described with reference to [FIG. 2] and to [FIG. 3].

In the context of the invention, a tumour is detected in a patient and a percutaneous tumour ablation procedure based on electromagnetic fields is programmed to remove it. In order to prepare the surgery, pre-procedural images of the tumour are collected by magnetic resonance imaging or by computed tomography scanning.

During a surgery 200, pre-procedural image data of one or more area(s) of interest including all or part of a tumour are received by the memory 3.

The pre-procedural image data may be stored directly in the memory 3 or be kept on another storage medium while awaiting for the surgery.

During the percutaneous tumour ablation procedure based on electromagnetic fields—for example a radiofrequency or microwave ablation, or an irreversible electroporation—the interventional radiologist inserts one or more electromagnetic stimulation needle(s) into the tumour.

Moreover, concomitantly with the percutaneous tumour ablation procedure, a cone beam computed tomography allows collecting current images. As explained before, an X-ray emitter is rotating around the patient and emits a conical beam which scans the target region. The X-rays are received by a receiver and current image data of one or more area(s) of interest including at least one electromagnetic stimulation needle are then generated.

During a surgery 210, the current image data obtained by cone beam computed tomography are received by the tool 5.

Besides the current image data, the tool 5 also receives pre-procedural image data originating from the memory 3.

During a surgery 220, the tool 5 implements a registration of current image data and of pre-procedural image data.

This registration is detailed hereinafter with reference to [FIG. 3] and may be split into two sub-operations: a sub-operation 300 of a nearest neighbour algorithm applied to at least one pair formed by a pre-procedural image and a current image, and then a sub-operation 310 of determining a geometric transformation allowing switching from a pre-procedural image into a current image.

In the description hereinafter, the registration of a pre-procedural image and of a current image is considered. Nonetheless, it should herein be understood that, advantageously, the tool 5 carries out a registration for several pairs formed respectively of a pre-procedural image and of a current image.

The sub-operation 300 corresponds to a “PatchMatch” type algorithm to establish a correspondence between the patches of the pre-procedural image and their nearest neighbours in the current image. This algorithm allows obtaining an table of correspondence, often referred to by the English acronym NNF standing for “Nearest Neighbour Field”. This table of correspondence is equivalent to a function which, at each patch of the pre-procedural image, associates a displacement vector which, starting from the patch of the current image occupying the same position as the considered patch of the procedural image, allows reaching the patch of the current image detected as being the nearest neighbour.

A “patch” herein refers to a local element of the domain of an image. In the case of a two-dimensional image, a patch is a square shaped local surface centred on a pixel. In the case of a three-dimensional image, a patch is a cubic shaped local volume centred on a voxel.

Thus, a patch may be decomposed into sub-elements which consist of pixels for a two-dimensional image or voxels for a three-dimensional image.

The algorithm “PatchMatch” can be split into three steps: an initialisation, a propagation and a random search. It is considered hereinafter that the pre-procedural image and the current image of the pair on which the tool 5 applies a registration are three-dimensional.

The initialisation consists in randomly associating each patch of the pre-procedural image with a patch of the current image.

It is considered hereinafter that the pre-procedural image and the current image have been decomposed into the same number of patches, so that a bijection could be randomly constructed between the pre-procedural image and the current image.

As explained before, the pre-procedural image and the current image are three-dimensional. Hence, each patch can be identified by three-dimensional coordinates of its central voxel. For example, any reference system with an origin O provided with an abscissa axis X, an ordinate axis Y and a dimension Z is used. For simplicity, it is possible to provide these axes with an arbitrary graduation so that each patch has integer coordinates.

It is considered hereinafter that the pre-procedural image and the current image are decomposed into M×N×P patches, where M, N and P are natural numbers. Each patch has coordinates (x,y,z), where x is an integer comprised between 1 and M, y is an integer comprised between 1 and N, and z is an integer comprised between 1 and P.

The propagation consists in examining each patch of the pre-procedural image one-by-one to improve the initial random correspondence with the current image. Typically, the order of propagation consists in examining the initial table of correspondence in the increasing direction of the axes, from left to right, from top to bottom and from front to back. Herein again, this is a convention and it is possible to proceed otherwise.

For each examined patch, all neighbouring patches already examined are considered. Except for the patches at the periphery of the pre-procedural image, each patch has exactly six neighbours, three of which have already been examined. Each of these three patches is associated with a displacement vector. The patch whose position corresponds to that of the patch being examined on the pre-procedural image is then considered on the current image. Four displacement vectors are applied to this patch on the current image: the displacement vector already associated, upon initialisation, to the examined patch and the three displacement vectors respectively associated with the neighbouring patches of the examined patch on the pre-procedural image. Thus, four patches are obtained on the current image: the patch already associated, upon initialisation, with the examined patch and three patches reached by application of each of the three remaining vectors. A distance is then calculated between the examined patch and each of these four patches and, among these four patches, that one which allows minimising this distance is selected. The patch examined is then associated with the corresponding displacement vector and therefore, in an equivalent manner, to the corresponding patch of the current image.

The propagation may be formalized as follows:

A patch examined on the pre-procedural image is centred on a voxel VPᵢ with the coordinates (xᵢ,yᵢ,zᵢ). The patch examined herein is not at the periphery of the pre-procedural image, thereby: xᵢ>1, yᵢ>1, zᵢ>1. The six neighbouring patches are centred on voxels with respective coordinates: (xᵢ-1,yᵢ,zᵢ), (xᵢ,yᵢ-1,zᵢ), (xᵢ,yᵢ,zᵢ-1), (xᵢ-1,yᵢ-1,zᵢ), (xᵢ-1,yᵢ,zᵢ-1) and (xᵢ,yᵢ-1,zᵢ-1). Among these six patches, three have already been examined and are centred on voxels whose respective coordinates are as follows: (xᵢ-1,yᵢ,zᵢ), (xᵢ,yᵢ-1,zᵢ) and (xᵢ,yᵢ,zᵢ-1).

The examined patch centred on VPᵢ has been associated, upon initialisation, with a displacement vector uᵢ(0.0.0). The patch to the left, already examined during the propagation, has been associated with a displacement vector uᵢ(−1,0,0). The patch at the top, already examined during the propagation, has been associated with a displacement vector uᵢ(0,−1,0). The patch at the front, already examined during the propagation, has been associated with a displacement vector uᵢ(0,0,−1).

Consider now, on the current image, the patch centred on the voxel VCᵢ, with the same coordinates (xᵢ,yᵢ,zᵢ) as the voxel VPᵢ on which the examined patch is centred. Each of the four displacement vectors is applied to the patch centred on the voxel VCᵢ to reach four voxels on the current image, therefore VCᵢ+uᵢ(0,0,0), VCᵢ+uᵢ(−1,0,0), VCᵢ+uᵢ(0,−1,0) and VCᵢ+uᵢ(0,0,−1). The distance between the patch centred on the examined voxel VPᵢ and each of the patches centred on the voxels thus reached is calculated. Then, as the displacement vector corresponding to the patch centred on the voxel VPᵢ, that one allowing minimising this distance is selected. Hence, by denoting u′ᵢ(0,0,0) the new displacement vector associated, during the propagation, with the patch centred on the voxel VPᵢ, we have:

$${u}_{i}^{′} ( 0 , 0 , 0 ) = arg {min}_{{u}_{i}} \{ D ( {VP}_{i} ; {VC}_{i} + {u}_{i} ( 0 , 0 , 0 ) ) , D ( {VP}_{i} ; {VC}_{i} + {u}_{i} ( - 1 , 0 , 0 ) ) , D ( {VP}_{i} ; {VC}_{i} + {u}_{i} ( 0 , - 1 , 0 ) ) , D ( {VP}_{i} ; {VC}_{i} + {u}_{i} ( 0 , 0 , - 1 ) ) \}$$

It is possible to define the distance D in different manners. In the context of the invention, a distance allowing favouring the alignments of contours between the pre-procedural image and the current image is selected. By considering \vec{∇}_{I} and \vec{∇}_{J} the respective gradients of the pre-procedural image I and of the current image J, the distance D(VPᵢ;VCᵢ₊ᵤᵢ) between a patch centred on the voxel VPᵢ on the pre-procedural image I and the corresponding patch potential centred on the voxel VCᵢ₊ᵤᵢ on the current image J may be defined as follows:

$$D ( {VP}_{i} ; {VC}_{i} + {u}_{i} ) = - \frac{{\int }_{Γ} \vert {\vec{∇}}_{I} ( {VP}_{i} + r ) \cdot {\vec{∇}}_{J} ( {VC}_{i} + {u}_{i} + r ) \vert dr}{{\int }_{Γ} {‖ {\vec{∇}}_{I} ( {VP}_{i} + r ) ‖}_{2} {‖ {\vec{∇}}_{J} ( {VC}_{i} + {u}_{i} + r ) ‖}_{2} d r}$$

where the local variable r is a displacement vector the variation of which enables VPᵢ₊ᵣ and VCᵢ₊ᵣ to cover the entirety of the considered patch ┌. In other words, any voxel of the considered patch on the pre-procedural image I, respectively on the current image J, can be reached from the central voxel VPᵢ, VCᵢ₊ᵤᵢ, respectively, with a displacement vector r. Thus, for any voxel V of the patch centred on the voxel VPᵢ of the pre-procedural image, respectively centred on the voxel VCᵢ₊ᵤᵢ of the current image, there is a displacement vector r such that V=VPᵢ₊ᵣ, respectively V=VCᵢ₊ᵤ_{ᵢ}₊ᵣ.

Finally, the random search consists in improving each displacement vector associated with a patch of the pre-procedural image by testing several candidates located at a decreasing distance exponentially from the nearest neighbour found during the propagation. Typically, for each patch of the current image, the propagation and then the random search are first applied, and we pass afterwards to the next patch.

The patch of the pre-procedural image centred on the voxel VPᵢ is considered again. Upon completion of the propagation, this patch is now associated with the displacement vector u′ᵢ(0,0,0) and therefore, in an equivalent manner, to the patch centred on the voxel VCᵢ+u′ᵢ(0,0,0) on the current image.

A set of candidate displacement vectors uₖ(0, 0, 0) in the following form is then tested:

$${u}_{k} ( 0 , 0 , 0 ) = {u}_{i}^{′} ( 0 , 0 , 0 ) + ω {β}^{k} {r}_{i}$$

where: —k is a natural number,
   ω is a maximum search radius,
   β is a fixed ratio between the sizes of search windows, typically equal to ½, and
   rᵢ is a random vector in [−1,1]3.

For each candidate uk_{(0,0,0)}, the distance between the patch centred on the voxel VPᵢ in the pre-procedural image and the patch centred on the voxel VCᵢ+uₖ(0,0,0) in the current image is measured. Then, as the displacement vector corresponding to the patch centred on the voxel VPᵢ, that one allowing minimising this distance is selected.

It should be noted that the propagation and the random search may be repeated several times to improve the table of correspondence between the pre-procedural image and the current image.

The sub-operation 310 corresponds to the actual registration of the current image data and of the pre-procedural image data. In practice, the registration consists in determining a geometric transformation allowing switching from the pre-procedural image I into the current image J. Such a geometric transformation allows aligning the pre-procedural image I and the current image J. In an equivalent manner, the current image J is registered on the pre-procedural image 1. It should be noted that the sub-operation 310 may be carried out alone, without the sub-operation 300, which is therefore optional but allows facilitating and accelerating the implementation of the registration.

Of course, it is also possible to register the pre-procedural image I on the current image J, and therefore determine a geometric transformation allowing switching from the current image I into the pre-procedural image J. It should be understood that, in the following description, the registration may be implemented by interchanging 1 and J.

To do so, an estimated criterion is first determined based on the respective gradients \vec{∇}_{I}, and \vec{∇}_{J} of the pre-procedural image I and of the current image J for a given geometric transformation T. Such a criterion may be estimated as follows by integrating over all of the voxels V of a patch ┌:

$$D ( T ) = {e}^{- C ( T )}$$

$$C ( T ) = \frac{{\int }_{Γ} \vert {\vec{∇}}_{I} ( T ( V ) ) \cdot {\vec{∇}}_{J} ( V ) \vert dV}{{\int }_{Γ} {‖ {\vec{∇}}_{I} ( T ( V ) ) ‖}_{2} {‖ {\vec{∇}}_{J} ( V ) ‖}_{2} d V}$$

Optionally, the tool 5 may apply a Sobel filter to the pre-procedural image I and to the current image J to determine the amplitudes M_{I}, M_{J} and orientations θᵢ, θᵢ of their respective gradients at a voxel V. The following expression is then obtained:

$$C ( T ) = \frac{{\int }_{Γ} \vert {ω}_{T} ( V ) \vert cos ( Δ {θ}_{T} ( V ) ) ‖ dV}{{\int }_{Γ} {ω}_{T} ( V ) d V}$$

where ω_{T}(V) and Δθ_{T}(V) are calculated as follows:

$${ω}_{T} ( V ) = {M}_{I} ( T ( V ) ) {M}_{J} ( V )$$

$$Δ {θ}_{T} ( V ) = {θ}_{I} ( T ( V ) ) - {θ}_{J} ( V )$$

Once the criterion D has been determined for any geometric transformation T, the tool 5 determines the geometric transformation allowing switching from the pre-procedural image I into the current image J by minimising an energy function dependent on this criterion.

For example, the energy function E is defined as follows on the image domain 0 which comprises all patches F:

$$E ( T ) = {\int }_{Ω} D ( T ) + \frac{α}{2} ( {‖ \vec{∇} u ‖}_{2}^{2} + {‖ \vec{∇} v ‖}_{2}^{2} + {‖ \vec{∇} w ‖}_{2}^{2} ) d V$$

where: —a is a weighting coefficient, and
   u, v and w are the components of the geometric transformation T.

As explained before, it is herein considered that the pre-procedural image and the current image are three-dimensional and, therefore, that the geometric transformation allowing switching from one into the other has three components. Nonetheless, the same method may be applied when the pre-procedural image and the current image are two-dimensional, in which case all it needs is to remove one of the components of the geometric transformation.

The tool 5 can minimise the energy function by solving a system of Euler-Lagrange equations obtained by deriving the energy function with respect to each component of the geometric transformation.

Such a system of equations may then be in the following form using a Laplacian operator A:

$$\{ \begin{matrix}\frac{\partial D}{\partial u} ( T ) - α Δ u = 0 \\ \frac{\partial D}{\partial v} ( T ) - α Δ v = 0 \\ \frac{\partial D}{\partial w} ( T ) - α Δ w = 0\end{matrix}$$

The value of each component of the geometric transformation may be calculated by iterations:

$$\{ \begin{matrix}{u}_{p + 1} = {u}_{p} + Δ {u}_{p} - {α}^{- 1} \frac{\partial D}{\partial u} ( {T}_{p} ) \\ {v}_{p + 1} = {v}_{p} + Δ {v}_{p} - {α}^{- 1} \frac{\partial D}{\partial v} ( {T}_{p} ) \\ {w}_{p + 1} = {w}_{p} + Δ {w}_{p} - {α}^{- 1} \frac{\partial D}{\partial w} ( {T}_{p} )\end{matrix}$$

The previous system corresponds to the p-th iteration. The sequences uₚ, vₚ and wₚ thus defined converge respectively towards the components of the pursued geometric transformation.

During an operation 230, the tool 5 returns cone beam computed tomography augmented image data. The augmented image data characterise an augmented image obtained by registration from a pair formed of a pre-procedural image and a current image. This image augmented by cone beam computed tomography has a quality close to the pre-procedural image and comprises an area of interest including at least one electromagnetic stimulation needle identified on the current image.

The cone beam computed tomography augmented image data may be stored in the memory 3.

The cone beam computed tomography augmented image data allows adjusting, where necessary, the position of the electromagnetic stimulation needles to ensure that they are properly inserted into the tumour without damaging a portion of the body or an organ located proximate thereto.

During an operation 240, the simulator 7 receives cone beam computed tomography augmented image data and electromagnetic stimulation parameters.

The cone beam computed tomography augmented image data may be received by the simulator 7 directly from the tool 5 or be recovered in the memory 3.

As explained before, the electromagnetic stimulation parameters may include the intensity of the electric current flowing in at least one electromagnetic stimulation needle. Preferably, these parameters include the intensity of the electric current flowing in each electromagnetic stimulation needle.

During an operation 250, the simulator 7 returns electromagnetic field simulation data.

To do so, the simulator 7 calculates an effective electrical conductivity of the tissues according to the intensity of the electric current flowing in each electromagnetic stimulation needle.

The case in which one or more pair(s) of electromagnetic stimulation needles are inserted into the tumour of the patient is considered hereinafter. This case is encountered in particular when the used percutaneous tumour ablation technique is irreversible electroporation. Indeed, irreversible electroporation functions in a bipolar mode—we also talk about a multi-bipolar mode when several pairs of needles are inserted—so that an electromagnetic field is created between each pair of electromagnetic stimulation needles. The electromagnetic stimulation needles forming a pair then act as active electrodes to deliver pulses of electromagnetic fields.

The effective electrical conductivity of the tissues may be calculated as follows for a pair of active electrodes—or electromagnetic stimulation needles —:

$${σ}_{eff} = \frac{{I}_{measured}}{{g}^{+} {\int }_{E {L}^{+}} {\partial }_{n} {u}^{+} ( x ) d x + {g}^{-} {\int }_{E L -} {\partial }_{n} {u}^{-} ( x ) d x}$$

where: —σ_{eff} is the effective electrical conductivity of the tissues,
   I_{measured} is the measured intensity of the electric current,
   EL⁺ and EL⁻ are the active electrodes,
   g⁺ and g⁻ are the respective electrical voltages of the active electrodes EL⁺ and EL⁻,
   x is a local variable flowing through each of the active electrodes EL⁺ and EL⁻, and
   ∂ₙu⁺, respectively ∂ₙu⁻, is the normal component of the gradient of the potential along the electrode EL⁺, respectively EL⁻.

Afterwards, the simulator 7 determines, from the effective electrical conductivity of the tissues, distribution data of the electromagnetic field. These data allow determining the distribution of the electromagnetic field in the area of the tumour and therefore, herein again, adjusting the position of the electromagnetic stimulation needles or varying the intensity of the electric current in each electromagnetic stimulation needle to carry out the percutaneous tumour ablation in the most efficient manner as possible.

The electromagnetic field simulation data may be stored in the memory 3. For example, the simulation data are stored in the DICOM (English acronym standing for “Digital Imaging and COmunication in Medicine”) format which is a conventional standard for data derived from medical imaging.

Finally, during an optional operation 260, the screen 9 receives electromagnetic field simulation data.

The electromagnetic field simulation data may be received by the screen 9 directly from the simulator 7 or be recovered in the memory 3.

Advantageously, the electromagnetic field simulation data comprise distribution data of the electromagnetic field, so that the screen 9 displays a distribution of the electromagnetic field in one or more area(s) of interest, and in particular in the area of the tumour.

The display of the distribution of the electromagnetic field on the screen 9 enables the interventional radiologist to have a visual feedback during the percutaneous tumour ablation procedure and to adjust the position of the electromagnetic stimulation needles.

Of course, feedbacks other than a visual feedback are possible so that the simulation data returned by the simulator 7 could be rendered in another form so as to inform the interventional radiologist on the position of the electromagnetic stimulation needles and on the electromagnetic field to which the area of the tumour is exposed.

Although the present disclosure has been described with reference to one or more examples, workers skilled in the art will recognize that changes may be made in form and detail without departing from the scope of the disclosure and/or the appended claims.

1. A medical assistance device comprising:
a memory arranged to receive pre-procedural image data of one or more area(s) of interest;
a tool arranged to receive current image data of one or more area(s) of interest including at least one electromagnetic stimulation needle obtained by cone beam computed tomography during a percutaneous ablation procedure based on electromagnetic fields, to implement registration of current image data and pre-procedural image data, and to return cone beam computed tomography augmented image data; and
a simulator arranged to receive cone beam computed tomography augmented image data and electromagnetic stimulation parameters, and to return electromagnetic field simulation data.

2. The device according to claim 1, further comprising a screen arranged to receive electromagnetic field simulation data, said simulation data comprising distribution data of the electromagnetic field, and to display a distribution of the electromagnetic field in one or more area(s) of interest.

3. A medical assistance method implemented by the device according to claim 1 and comprising:
receiving the pre-procedural image data of the one or more area(s) of interest including all or part of a tumour,
receiving the current image data of the one or more area(s) of interest including at least one electromagnetic stimulation needle obtained by cone beam computed tomography during the percutaneous ablation procedure based on electromagnetic fields,
implementing the registration of the current image data and the pre-procedural image data,
returning the cone beam computed tomography augmented image data,
receiving the cone beam computed tomography augmented image data and electromagnetic stimulation parameters, and
returning the electromagnetic field simulation data.

4. The method according to claim 3, wherein the pre-procedural image data are obtained by magnetic resonance imaging or computed tomography scanning.

5. The method according to claim 3, wherein the pre-procedural image data and the current image data relate to three-dimensional images of the one or more area(s) of interest.

6. The method according to claim 3, wherein the registration comprises an initial operation of implementing a nearest neighbour algorithm applied to at least one pair formed of a pre-procedural image and a current image.

7. The method according to claim 6, wherein the pre-procedural image and the current image are respectively decomposed into a set of local elements, named patches, and wherein the nearest neighbour algorithm comprises a propagation during which a distance between a patch of the pre-procedural image and a patch of the current image is calculated as follows:
$$D ( VP ; VC ) = - \frac{{\int }_{Γ} \vert {\vec{∇}}_{I} ( VP + r ) \cdot {\vec{∇}}_{J} ( VC + r ) \vert dr}{{\int }_{Γ} {‖ {\vec{∇}}_{I} ( VP + r ) ‖}_{2} {‖ {\vec{∇}}_{J} ( VC + r ) ‖}_{2} d r}$$
where:
I is a pre-procedural image and J is a current image,
VP and VC are respective centres of the patches of the pre-procedural image I and of the current image J,
r is a local variable corresponding to a displacement vector whose variation enables VP+r and VC+r to cover the entirety of the considered patch r, and
\vec{∇}ᵢ and \vec{∇}ⱼ are respective gradients of the pre-procedural image and the current image.

8. The method according to claim 3, wherein the registration comprises determining a geometric transformation allowing switching from a pre-procedural image into a current image, or vice versa, by minimizing an energy function depending on an estimated criterion based on respective gradients of the pre-procedural image and of the current image.

9. The method according to claim 8, wherein the energy function is defined as follows:
$$E ( T ) = {\int }_{Ω} D ( T ) + \frac{α}{2} ( {‖ \vec{∇} u ‖}_{2}^{2} + {‖ \vec{∇} v ‖}_{2}^{2} + {‖ \vec{∇} w ‖}_{2}^{2} ) d \vec{r}$$
where: —T is a geometric transformation,
E is the energy function defined on an image domain Q,
D is the estimated criterion,
α is a weighting coefficient,
u, v and w are components of the geometric transformation T.

10. The method according to claim 9, wherein the energy function is minimised by solving a system of Euler-Lagrange equations obtained by deriving the energy function with respect to each component of the geometric transformation, the value of each component being calculated by iteration.

11. The method according to claim 8, wherein the criterion is estimated as follows:
D(T)=e^{-C(T)}
$$C ( T ) = \frac{{\int }_{Γ} \vert {\vec{∇}}_{I} ( T ( V ) ) \cdot {\vec{∇}}_{J} ( V ) \vert dV}{{\int }_{Γ} {‖ {\vec{∇}}_{I} ( T ( V ) ) ‖}_{2} {‖ {\vec{∇}}_{J} ( V ) ‖}_{2} dV}$$
where: —T is a geometric transformation,
D is the calculated criterion on a patch F,
I is a pre-procedural image and J is a current image,
V is a sub-element of the patch F, and
\vec{∇}_{I} and \vec{∇}_{J} are respective gradients of the pre-procedural image and of the current image.

12. The method according to claim 8, wherein a Sobel filter is applied to the pre-procedural image and to the current image to determine an amplitude and an orientation of their respective gradients.

13. The method according to claim 3, wherein the electromagnetic stimulation parameters include an intensity of the electric current flowing in at least one electromagnetic stimulation needle, and wherein the simulation data comprise distribution data of the electromagnetic field, said distribution of the electromagnetic field being determined from an effective electrical conductivity of tissues in one or more area(s) of interest calculated according to the intensity of the electric current flowing in said at least one electromagnetic stimulation needle.

14. The method according to claim 13, wherein one or more pair(s) of electromagnetic stimulation needles are used to implement the percutaneous ablation procedure based on electromagnetic fields, so that two electromagnetic stimulation needles of the same pair act as active electrodes, and wherein the effective electrical conductivity of the tissues is calculated as follows for such a pair of active electrodes:
$${σ}_{eff} = \frac{{I}_{measured}}{{g}^{+} {\int }_{E {L}^{+}} {\partial }_{n} {u}^{+} ( x ) dx + {g}^{-} {\int }_{E L -} {\partial }_{n} {u}^{-} ( x ) dx}$$
where:
σ_{eff} is the effective electrical conductivity of the tissues,
I_{measured} is the measured intensity of the electric current,
EL⁺ and EL⁻ are the active electrodes,
g⁺ and g⁻ are respective electrical voltages of the active electrodes EL⁺ and EL⁻,
x is a local variable flowing through each of the active electrodes EL⁺ and EL⁻, and
∂ₙu⁺, respectively ∂ₙu⁻, is a normal component of a gradient of the potential along the electrode EL⁺, respectively EL⁻.

15. A non-transitory computer readable medium comprising a computer program stored thereon comprising instructions for implementing the method according to claim 3, when said instructions are executed by at least one processor.
